(19) United States
(12) Patent Application Publication
(10) Pub. No.: US 2024/0352443 A1
(43) Pub. Date: Oct. 24, 2024

(54) METHOD FOR GENERATING DOUBLE-STRANDED NUCLEIC ACID

(71) Applicant: DNA Script, Le Kremlin-Bicêtre (FR)

(72) Inventors: Sandrine CRETON, Le Kremlin-Bicêtre (FR); Florence MAHE, Le Kremlin-Bicêtre (FR)

(21) Appl. No.: 18/686,319

(22) PCT Filed: Jul. 22, 2022
(86) PCT No.: PCT/EP2022/070699

§ 371 Date: Feb. 23, 2024

(30) Foreign Application Priority Data
Aug. 23, 2021 (EP) EP21192541

(51) Int. Cl. C12N 15/10; C12N 9/00; C12N 9/22

(52) U.S. Cl. CPC C12N 15/1031; C12N 9/22; C12N 9/93; C12Y 605/01001

(57) ABSTRACT

The present invention relates to a method for generating a double-stranded nucleic acid having a predetermined sequence and a method for producing a double-stranded polynucleotide of interest. The invention further relates to a kit for the implementation of said methods.

The present invention relates to a method for generating double-stranded nucleic acid(s) having predetermined sequence(s) from oligonucleotides.

### BACKGROUND

The synthesis of double-stranded nucleic acid is of fundamental importance to the molecular cloning and analysis of eukaryotic genes. Recent progress in DNA synthesis has now made it possible to rapidly synthesize short single-stranded oligonucleotides. After synthesis of short single-stranded oligonucleotides, it is necessary to assemble these oligonucleotides to generate a long polynucleotide. Oligonucleotides assembly methods for producing long double-stranded DNA (herein after “dsDNA”) have been reported in the prior art. For example, Pengpumkiat et al (PLOS ONE 11(3): e0149774, March 1, 2016) disclosed a method for synthetizing a long double-stranded oligonucleotide on magnetic beads from short single-stranded oligonucleotide through successive annealing and ligation processes. In this method, the first short single-stranded oligonucleotide was immobilized onto magnetic beads via streptavidinbiotin binding. Next, single-stranded oligonucleotide fragments were added successively through annealing and ligation to form a complete dsDNA molecule. Then, the synthesized dsDNA was amplified through PCR. U.S. Pat. No. 9,295,965 also described a method for synthesizing nucleic acid having a predefined sequence on a solid support. In this method, single-stranded oligonucleotides are firstly bound onto a solid support to provide a beginning primer for successive hybridizations of a series of oligonucleotides with predefined sequences to construct a longer polynucleotide. These oligonucleotides are then assembled together by ligation. However, these methods have several drawbacks. Both methods need the use of a solid support and rely on polymerase amplification to amplify full-length polynucleotide. Moreover, further gel purification or affinity purification are needed, rendering automatization of the method difficult.

There is therefore a need for an improved method which can produce efficiently and cost-effectively double-stranded nucleic acids with none or low error rate.

### SUMMARY OF THE INVENTION

By working on dumbbell-shaped DNA, the Inventors have discovered that dumbbell-shaped DNA molecule can be used as intermediate molecule for generating a linear double-stranded nucleic acid with a predetermined sequence. More particularly, they developed a method, wherein single-stranded oligonucleotides of predetermined sequences are assembled to form a dumbbell-shaped nucleic acid molecule, before to generate a linear double-stranded nucleic acid with predetermined sequence. Advantageously, this method allows to circumvent gel purification, affinity purification and/or polymerase amplification. This method may be easily automated. Moreover, no solid support is required to implement the method of the present invention, leading to more cost-efficient and time-saving production compared to prior known methods.

The method combines a specific closed linear dsDNA feature with a specific successive order of the subsequent enzymatic steps: 1/ Design closed linear dsDNA, 2/ ligate closed structure, 3/ digest any linear structure (i.e. isolate successful assemblies), 4/ Revert the loop, return to pure full-length standard linear dsDNA.

In a first aspect, the present invention provides a method for generating a double-stranded nucleic acid having a predetermined sequence, said method comprising the following steps:
(a) providing (i) a plurality of single-stranded oligonucleotides, (ii) a first hairpin-forming oligonucleotide comprising a single-stranded overhang and (iii) a second hairpin-forming oligonucleotide comprising a single-stranded overhang,
the sequence of each single-stranded oligonucleotide being at least partially complementary with the sequence of at least one other single-stranded oligonucleotide of said plurality, and/or with the sequence of the single-stranded overhang of the first or second hairpin-forming oligonucleotide,
(b) reacting said plurality of single-stranded oligonucleotides and the first and second hairpin-forming oligonucleotides with a ligase to form a closed double-stranded nucleic acid with two single-stranded oligonucleotide loops in a reaction medium,
(c) subjecting the reaction product of step (b) to enzymatic, physical and/or chemical means to eliminate single-stranded oligonucleotide loops, intermediate products, unassembled single-stranded oligonucleotides and/or unassembled hairpin-forming oligonucleotides,
(d) optionally performing error correction step(s),
(e) optionally recovering the double-stranded nucleic acid.

In a further embodiment of the invention, the method comprises the following steps:
(a) providing (i) a plurality of single-stranded oligonucleotides, (ii) a first hairpin-forming oligonucleotide comprising a single-stranded overhang and (iii) a second hairpin-forming oligonucleotide comprising a single-stranded overhang,
the sequence of each single-stranded oligonucleotide being at least partially complementary with the sequence of at least one other single-stranded oligonucleotide of said plurality, and/or with the sequence of the single-stranded overhang of the first or second hairpin-forming oligonucleotide,
(b) reacting said plurality of single-stranded oligonucleotides and the first and second hairpin-forming oligonucleotides with a ligase to form a closed double-stranded nucleic acid with two single-stranded oligonucleotide loops in a reaction medium,
(c1) subjecting the reaction product of step (b) to an exonuclease to degrade intermediate products, unassembled single-stranded oligonucleotides and/or unassembled hairpin-forming oligonucleotides,
(c2) subjecting the reaction product of step (c1) to a nuclease to digest the single-stranded oligonucleotide loops of said closed double-stranded nucleic acid;
(d) optionally performing error correction step(s),
(e) optionally recovering the double-stranded nucleic acid.

Another aspect of the present invention relates to a method for producing a double-stranded polynucleotide of interest. Said method comprises the following steps:
generating at least two double-stranded nucleic acids corresponding to at least two different fragments of said polynucleotide by the aforementioned method of the invention,
linking said fragments according to a predetermined order by use of a DNA ligase and/or polymerase to produce said polynucleotide.

The present invention also relates to a kit for the implementation of the methods described above, comprising:
a plurality of single-stranded oligonucleotides of predetermined sequences, a first hairpin-forming oligonucleotide comprising a single-stranded overhang of predetermined sequence and a second hairpin-forming oligonucleotide comprising a single-stranded overhang of predetermined sequence;
Optionally a first enzymatic reagent and/or a first reaction medium to be used for assembling the single-stranded oligonucleotides and a first and second hairpin-forming oligonucleotides into a closed double-stranded nucleic acid,
Optionally a second enzymatic reagent and/or a second reaction medium to be used for eliminating single-stranded oligonucleotide loops, intermediate products, unassembled single-stranded oligonucleotides and/or unassembled hairpin-forming oligonucleotides.

### BRIEF DESCRIPTION OF THE DRAWINGS

FIG. 1A diagrammatically illustrates a method of the present invention for generating a double-stranded nucleic acid having a predetermined sequence.

FIG. 1B illustrates further steps for producing a double-stranded polynucleotide of interest from double-stranded nucleic acids, according to an embodiment of the present invention.

FIG. 2 diagrammatically illustrates an embodiment of a method of the present invention for generating a double-stranded nucleic acid having a predetermined sequence, wherein step (b) is performed by two different ligases.

FIG. 3 diagrammatically illustrates some steps of a method of the present invention according to a particular embodiment, wherein by-products of step (b) are eliminated.

FIG. 4 diagrammatically illustrates error-correction steps that may be implemented in a particular embodiment of a method of the present invention.

FIG. 5A-C illustrate an embodiment of a loop assembly method of the present invention wherein a 820 base pairs dsDNA fragment was generated.

FIG. 6A-B diagrammatically illustrate some steps of a method of the present invention according to a particular embodiment, wherein removal of the loops is performed with nuclease P1.

FIG. 7A-B illustrate how assembly of oligonucleotides into dsDNA allows the creation of de novo DNA sequences, without preexisting template.

FIG. 8 (Table 1) lists oligonucleotides synthesized in example 1.

FIG. 9 (Table 2) lists oligonucleotides composing part #1 and part #2 of example 3.

### DESCRIPTION OF THE INVENTION

The present invention relates to a method for generating a double-stranded nucleic acid having a predetermined sequence.

### Definitions

The present disclosure will be best understood by reference to the following definitions.

As used herein, the term “nucleic acid” encompasses deoxyribonucleic acid (DNA) or ribonucleic acid (RNA) which may comprise both natural and non-natural nucleotides. Natural nucleotides are, for examples, deoxyadenosine, deoxycytidine, deoxyguanosine, or deoxythymidine for DNA or their ribose counterparts for RNA. Non-natural nucleotide that can be cited include modified bases, sugars, or internucleosidic linkages. Non-naturally occurring nucleotides may also include Peptide Nucleic Acid (PNA) phosphorothioate internucleosidic linkages, bases containing linking groups permitting the attachment of labels, such as fluorophores, or haptens, and the like. A double-stranded nucleic acid may exist as double-stranded DNA or RNA, an RNA/DNA hybrid or an RNA/DNA copolymer, wherein the term “copolymer” refers to a single nucleic acid strand that comprises both ribonucleotides and deoxyribonucleotides.

The term “double-stranded nucleic acid” as used herein, and unless otherwise specified, refers to an open linear double-stranded nucleic acid with two free ends, by opposition to a closed double-stranded nucleic acid. Such double-stranded nucleic acid comprises two nucleic acid strands substantially complementary to each other along the entire length of the strands.

The term “closed double-stranded nucleic acid” as used herein refers to a closed linear double-stranded nucleic acid. In other words, the closed double-stranded nucleic acid has no free end.

The term “double-stranded” (“ds”) refers to a pair of nucleic molecules that exists in a hydrogen-bonded, double-helical structure.

At least one nucleotide of the closed double-stranded nucleic acid can have a tag for purifying DNA. Such a tag can be, for example, a biotin moiety. The tag can be attached to one of the nucleotides of the closed double-stranded nucleic acid by click chemistry or any other chemical reactions. More specifically, the tag can be attached to one of the nucleotides forming one the loops of the closed double-stranded nucleic acid.

As used herein, the term “complementary” in the context of the nucleotides refers to the hybridization or Watson & Crick base pairing between nucleotides. Complementary nucleotides are, generally, A and T (or A and U), or C and G. Two single-stranded oligonucleotides which are substantially complementary will hybridize to each other under stringent conditions. The term “substantially complementary” in the context of oligonucleotides or nucleic acids as used herein refers both to complete complementarity of two nucleic acid strands as well as complementarity sufficient to achieve the desired binding of two nucleic acid strands. Two single-stranded RNA or DNA molecules are substantially complementary when the nucleotides of one strand, optimally aligned and compared, pair with at least about 80% of the nucleotides of the other strand, usually at least about 90% to 95%, and more preferably from about 98 to 100%. Stringent hybridization conditions are well known in the art and defined by salt concentrations and hybridization temperature. The terms “hybridization,” “hybridize,” “anneal” or “annealing” as used herein refer to the ability, under appropriate conditions, for nucleic acids having substantial complementary sequences to bind to one another by Watson & Crick base pairing.

The terms “predetermined sequence” or “predefined sequence” as used herein refer to a nucleic acid sequence which is known and identified.

### Oligonucleotides of Step (a)

According to the present invention, a closed double-stranded nucleic acid is first formed by use of single stranded oligonucleotides and hairpin-forming oligonucleotides, that are assembled to form a dumbbell nucleic acid.

More particularly, the method of the present invention proposes to use (i) a plurality of single-stranded oligonucleotides (also designed below as ss-oligonucleotides), (ii) a first hairpin-forming oligonucleotide comprising a single-stranded overhang and (iii) a second hairpin-forming oligonucleotide comprising a single-stranded overhang to form a closed double-stranded nucleic acid, i.e., a dumbbell nucleic acid.

The terms “dumbbell nucleic acid” or “dumbbell-shaped nucleic acid” refer to a double-stranded nucleic acid with closed single-stranded oligonucleotide loops on each end.

The term “oligonucleotide” as used herein refers to oligodeoxyribonucleotide, which is solely composed of deoxyribonucleotides, or to oligoribonucleotide, which is solely composed of ribonucleotides, or to a nucleic acid comprising both deoxyribonucleotides and ribonucleotides.

Examples of deoxyribonucleotide can be selected from dATP, dGTP, dTTP, dUTP, dCTP, dITP and one of their association.

Examples of ribonucleotide can be selected from rATP, rGTP, rUTP, rCTP, rTMPrITP.

The term “hairpin-forming oligonucleotide” as used herein refers to a single-stranded oligonucleotide which comprises two sequences respectively at 5′ and 3′ terminal regions that are inverted repeats to form a double-stranded stem under adequate annealing conditions, a non-self-complementary central region to form a single-stranded loop and a 5′ or 3′ terminal non-self-complementary region, also called “single-stranded overhang”.

Said single-stranded oligonucleotides and hairpin-forming oligonucleotides may comprise natural nucleotides and/or non-natural nucleotides.

Each ss-oligonucleotide of said plurality and the single-stranded overhang of each hairpin-forming oligonucleotides are designed in a way that the nucleic acid sequence of each ss-oligonucleotide is at least partially complementary with the nucleic acid sequence of at least one other ss-oligonucleotide of said plurality, and/or with the nucleic acid sequence of the single-stranded overhang of the first or second hairpin-forming oligonucleotide. That is to say that at least a nucleic acid segment of each ss-oligonucleotide is complementary with at least a nucleic acid segment of another ss-oligonucleotide and/or with at least a nucleic acid segment of the overhang of the first or second hairpin-forming oligonucleotide.

Advantageously, each complementary segment of a single-stranded oligonucleotide comprises at least 5 nucleotides, particularly from 5 to 35, from 5 to 30, particularly from 10 to 30, from 10 to 25, from 15 to 30, more particularly from 15 to 25 nucleotides.

In a particular embodiment, each ss-oligonucleotide of the plurality comprises two segments of sequence, each segment being complementary with a segment of sequence of another ss-oligonucleotides of the same plurality and/or with all or part of the single-stranded overhang of the first or second hairpin-forming oligonucleotide.

In a more particular embodiment, the sequence of each ss-oligonucleotide of said plurality consists of two segments, each segment being complementary with a segment of the sequence of another ss-oligonucleotide of said plurality, and/or with the full sequence of the single-stranded overhang of the first or second hairpin-forming oligonucleotide. Advantageously, each of the two segments of a ss-oligonucleotide comprises at least 5 nucleotides, particularly from 5 to 35, from 5 to 30, particularly from 10 to 30, from 10 to 25, from 15 to 30, more particularly from 15 to 25 nucleotides.

In some embodiments, said plurality of single-stranded oligonucleotides comprises from 2 to 100 single-stranded oligonucleotides, preferably from 3 to 60 single-stranded oligonucleotides, from 3 to 50 single-stranded oligonucleotides, from 3 to 40 single-stranded oligonucleotides, from 3 to 30 single-stranded oligonucleotides, from 3 to 20 single-stranded oligonucleotides.

In some embodiments, each single-stranded oligonucleotide comprises from 10 to 100 nucleotides, particularly from 10 to 90 nucleotides, from 10 to 80 nucleotides, from 10 to 70 nucleotides, from 10 to 60 nucleotides, from 10 to 50 nucleotides, from 10 to 40 nucleotides, from 10 to 30 nucleotides, from 10 to 20 nucleotides, from 15 to 35 nucleotides, from 15 to 30 nucleotides, more particularly from 20 to 30 nucleotides.

In some embodiments, the single-stranded overhang of the first and/or second hairpin-forming oligonucleotide comprises from 5 to 35 nucleotides, from 5 to 30 nucleotides, particularly from 10 to 30 nucleotides, from 10 to 25 nucleotides, more particularly from 15 to 25 nucleotides.

In some embodiments, the double-stranded stem of the first and/or second hairpin-forming oligonucleotides comprises from 3 to 20 base pairs, from 5 to 20 base pairs, from 5 to 15 base pairs, particularly from 5 to 10 base pairs.

In some embodiments, the single-stranded loop of the first and/or second hairpin-forming oligonucleotides comprises from 1 to 20, particularly from 1 to 15 nucleotides, from 1 to 10, more particularly from 1 to 9 nucleotides, from 1 to 8 nucleotides, from 1 to 7 nucleotides, from 1 to 6 nucleotides, from 1 to 5 nucleotides, from 2 to 10 nucleotides, from 2 to 9 nucleotides, from 2 to 8 nucleotides, from 2 to 5 nucleotides.

In some embodiments, the single-stranded loop of the first and/or second hairpin-forming oligonucleotides may comprise specific sequences that are recognized by endonuclease, or modified nucleotides that can be specifically cleaved by endonuclease (such as dU/USER, oxoG & FpG or endonuclease V . . . )

The single-stranded oligonucleotides and the single-stranded overhang of the first and second hairpin-forming oligonucleotide have predetermined and known nucleic acid sequences.

They can be designed and synthesized by any conventional method, for example by chemical synthesis based on solid-phase phosphoramidite chemistry described by Adams et al. (1983, J. Amer. Chem. Soc., 105, 661) and Froehler et al. (1983, Tetrahedron Lett., 24, 3171) or by enzymatic synthesis. Template-independent enzymatic oligonucleotide synthesis methods are for example described in detail in WO 2015/159023, WO 2017/216472, U.S. Pat. Nos. 5,436,143, 5,763,594, Jensen et al (Biochemistry, 57:1821-1832 (2018)), or Mathews et al (Organic & Biomolecular Chemistry, DOI: 0.1039/c60b01371f (2016)); Schmitz et al (Organic Lett., 1(11): 1729-1731 (1999)).

Advantageously, the single-stranded oligonucleotides and/or hairpin-forming oligonucleotides used in the present invention comprise a phosphorylated 5′ terminal and a free 3′-hydroxyl group. 5′ phosphate group can be pre-existing from former enzymatic activity. For example, when oligonucleotides are synthesized by an enzymatic method as described in WO 2017/216472, enzymatic synthesized oligonucleotides are released from a solid support with an enzymatic cleavage step that leaves a 5′ phosphate group on the oligonucleotide. A 5′ phosphate group may also be added by a kinase treatment (e.g. T4 polynucleotide kinase) or by a chemical treatment. S-triphenylmethyl O-methoxymorpholinophosphinyl 2-mercaptoethanol can be (Connolly, 1987); 2-cyanoethyl 3-(4,4′-dimethoxytrityloxy)-2,2-used di(efhoxycarbonyl)propyl-1 N, N-diisopropyl phosphoramidite (Guzaev et al, Tetrahedron, 1995; Horn and Urdea, 1986)

### Assembling Step (b)

According to the method of the present invention, the plurality of single-stranded oligonucleotides and the first and second hairpin-forming oligonucleotides are assembled together to form a closed double-stranded nucleic acid with two single-stranded oligonucleotide loops.

Generally speaking, this step of assembling comprises annealing (or pairing) the single-stranded oligonucleotide and ligating said pairing oligonucleotides. Pairing and ligating may be performed simultaneously or sequentially, in a same or in different reaction medium.

In a particular embodiment, the plurality of single-stranded oligonucleotide and the first and second hairpin-forming oligonucleotides are submitted to annealing, or hybridization, to obtain spontaneous pairing of the complementary nucleic acid sequences. Since each single-stranded oligonucleotide and single-stranded overhang of the hairpin-forming oligonucleotides have predetermined sequences, the oligonucleotides are annealed in a determined order to form a nucleic acid structure that is suitable to create a closed double-stranded nucleic acid of predetermined sequence.

According to the present invention, this step of annealing the plurality of single-stranded oligonucleotide is performed in solution, in a reaction medium. The single-stranded oligonucleotides and hairpin-forming oligonucleotides are contacted in the reaction medium at temperature and pH suitable to allow the pairing.

The annealing may be implemented according to any conventional method such as heat denaturation followed with incubation at annealing temperature in presence of mild salt conditions.

In an embodiment, annealing is performed in a standard annealing buffer, or reaction buffer adapted to each kind of ligase The annealing buffer comprises salts at low concentration, i.e the salts concentration is lower than 200 mM, a degradation inhibitor such as EDTA, and buffer species to keep pH between 7 and 8.

The nucleic acid structure obtained after annealing is submitted to ligation to seal the nicks and eventually gaps of said nucleic acid structure.

According to a particular embodiment, the ligation is performed by chemical reaction(s), e.g., reaction(s) involving phosphorothioate derivatives, CNBr or other reactive groups.

According to another particular embodiment, the ligation is implemented with enzymes, particularly DNA or RNA ligases able to catalyze the ligation of a 5′ phosphoryl-terminated nucleic acid to a 3′ hydroxyl-terminated nucleic acid through the formation of a 3′→5′ phosphodiester bond.

For instance, the ligase may be any thermostable ligase that is able to recognize two adjacent nucleotides bridged by a complementary DNA/RNA strand, particularly selected from T4 ligase, Ampligase, HiFi Taq ligase, Pfu DNA ligase, and/or 9° north ligase. Said embodiment is preferred when there is no gap between two adjacent nucleotides to be bridged.

In a particular embodiment, ligation(s) may be performed simultaneously or successively with at least two different types of enzymes, such a DNA/RNA ligase and a DNA/RNA polymerase. Said embodiment allows to fill the gap between two adjacent nucleotides in the presence of complementary sequence and then to seal the nick by a ligase. Preferably, said gap is a gap of at most 15 nucleotides between two adjacent nucleotides belonging to two oligonucleotides to be bridged.

In a particular embodiment, step (b) is implemented with at least two different ligases. Since each ligase may have substrate specificity and may lead to sequence bias, use of more than one ligase can be advantageous and provide a better ligation performance and efficiency. Different ligases can be used simultaneously or successively. Alternatively, same ligase may be used twice, successively.

More generally, the skilled person is able to select the appropriate ligases and/or polymerase, depending on sequences to ligate.

In a particular embodiment, at least two steps of ligation are implemented successively with different ligases. For instance, a first ligation is performed with HiFi Taq ligase and a second ligation is performed with Ampligase. For another example, a first ligation is performed with HiFi Taq ligase and a second ligation is performed with T4 DNA ligase. These successive ligations may be performed in the same reaction medium, i.e., without elimination of any previous enzyme, or in different and successive reaction mediums.

In a particular embodiment, ligation is performed by use, simultaneously, of several ligases, such as HiFi Taq ligase and/or Ampligase and/or HiFi Taq ligase and/or T4 DNA ligase.

Advantageously, step (b) is performed under controlled conditions, e.g. light, heat, pH, and presence of specific reagents, optimized to favor annealing and/or ligation.

Both annealing and ligation may be optimized by use of suitable reaction medium(s).

In a particular embodiment, annealing and ligation are performed in a same reaction medium. Alternatively, annealing and ligation are performed in different reaction mediums.

For example, the annealing reaction medium may contain 10 mM tris pH 7.5, 50 mM NaCl 1 mM EDTA.

For example, the ligation reaction medium may contain at least salt which are essential for enzyme activity, such as Mg²⁺ or other buffers such as 20 mM Tris HCl pH 8.5, 150 mM KCl, 10 mM MgCl2, 10 mM DTT, 1 mM NAD, 0.1% triton X-100

At the end of step (b), the reaction product in the reaction medium comprises closed double-stranded nucleic acid and by-products, e.g., unassembled single-stranded oligonucleotides, unassembled hairpin-forming oligonucleotides and/or intermediate products. As used herein, “intermediate products” refer to partially assembled and/or partially sealed double-stranded nucleic acids with two single-stranded oligonucleotide loops. Partially assembled intermediate products may consist in open nucleic acids or hairpin form nucleic acids. Partially sealed intermediate products contain at least one nick.

The closed double-stranded nucleic acid obtained at the end of step (b) comprises a double-stranded central part and single-stranded oligonucleotide loops at the two ends of said central part.

In some embodiments, said central part has a length from 50 to1000 base pairs, from 50 to 900 base pairs, from 100 to 900 base pairs, from 100 to 800 base pairs, from 100 to 700 base pairs, from 100 to 600 base pairs, from 100 to 500 base pairs, from 150 to 800 base pairs, from 200 to 700 base pairs, particularly from 200 to 600 base pairs, from 200 to 500 base pairs.

In some embodiments, each of said single-stranded oligonucleotide loop comprises from 1 to 20 nucleotides, particularly from 1 to 15 nucleotides, from 1 to 10 nucleotides, more particularly from 1 to 9 nucleotides, from 1 to 8 nucleotides, from 1 to 7 nucleotides, from 1 to 6 nucleotides, from 1 to 5 nucleotides, from 2 to 10 nucleotides, from 2 to 9 nucleotides, from 2 to 8 nucleotides, from 2 to 5 nucleotides.

### Elimination Step (c)

The reaction product issued from step (b) is further submitted to step (c) in order to eliminate the two single-stranded oligonucleotide loops of the closed double-stranded nucleic acid and/or the by-products of the reaction product.

The terms “eliminate” or “elimination” as used herein with reference to step (c) encompass separation, degradation, digestion, cleavage and/or hydrolyze. Generally speaking, any means suitable to suppress by-products and the single-stranded oligonucleotide loops of the closed double-stranded nucleic acid are encompassed.

For instance, these eliminations may be performed with chemical, physical and/or enzymatic means or by combinations thereof.

According to a particular embodiment, the envisioned eliminations are performed with chemical means. For instance, CeIV can induce DNA phosphodiester bond hydrolysis and lanthanide III can induce RNA phosphodiester bond hydrolysis (Komiyama et al, 1999; Matsumura et al, 1997).

According to a particular embodiment, the envisioned eliminations are performed with physical means. For instance, photocleavage of a modified nucleotide that is light-sensitive or single-strand photocleavage (Blacker, 1997, Fernandez-Saiz, 1999).

According to a particular embodiment, the envisioned eliminations are performed by use of degrading enzymes, for example exonucleases and/or nucleases, or sequence-specific endonuclease such as type IIS enzymes or endonuclease that recognize modified nucleotide (for example USER/dU; FpG/oxo-guanine. . . . )

In a particular embodiment, a forementioned step (c) is performed by contacting the reaction product of step (b) with at least one exonuclease and/or at least one nuclease.

According to an embodiment, the reaction product issued from step (b) is contacted with at least one exonuclease to degrade unassembled monomers and intermediate products. A skilled person knows that exonucleases may be sequence specific and/or structure specific and accordingly can choose or combine suitable exonucleases according to the nucleic acid to be degraded. Examples of exonucleases which can be used in the method of the prevent invention include, without limitation, exonuclease V, exonuclease T5, nuclease BAL-31, exonuclease III, Lambda exonuclease, exonuclease VII, or any combination thereof, such as combination of exonuclease III and Lambda exonuclease, or combination of exonuclease VII and exonuclease III.

According to an embodiment, the reaction product issued from step (b) is contacted with at least one single-stranded nuclease to degrade single-stranded loops of the closed double-stranded nucleic acid. A skilled person knows that a single-stranded nuclease may be sequence specific and/or structure specific and accordingly can choose or combine suitable nucleases according to the nucleic acid to be degraded. Examples of a single-stranded specific nuclease that can be used in the method of the prevent invention include, without limitation, nuclease P1, Mung Bean nuclease, or nuclease S1.

In a more particular embodiment, step (c) is performed by contacting the reaction product of step (b) first with an exonuclease, and secondly with a nuclease.

In a particular embodiment, step (c) comprises the following steps:
(c1) subjecting the reaction product of step (b) to an exonuclease to degrade the intermediate products, unassembled single-stranded oligonucleotides and/or unassembled hairpin-forming oligonucleotides, and
(c2) subjecting the reaction product of step (c1) to a nuclease to digest the single-stranded oligonucleotide loops of the closed double-stranded nucleic acid.

At the end of step (c1), the remained reaction product consists in the closed double-stranded nucleic acid and nucleotides released by exonuclease.

In another particular embodiment, step (c) is performed by contacting the reaction product of step (b) simultaneously with an exonuclease and a nuclease to eliminate two single-stranded oligonucleotide loops, the intermediate products, unassembled single-stranded oligonucleotides and/or unassembled hairpin-forming oligonucleotides.

The enzymatic degradation may be optimized by controlling the reactions conditions, e.g. light, heat, pH, addition of specific reagents, reactional buffer, etc. Advantageously, the enzymatic degradation is conducted at the optimum pH and temperature of the corresponding enzyme. The person skilled in the art is able to adapt the reactions conditions to the enzyme(s) involved.

The linear double-stranded nucleic acid resulting from step (c) may have a length from 50 to 1000 base pairs, from 50 to 900 base pairs, from 100 to 900 base pairs, from 100 to 800 base pairs, from 100 to 700 base pairs, from 100 to 600 base pairs, from 100 to 500 base pairs, from 150 to 800 base pairs, from 200 to 700 base pairs, particularly from 200 to 600 base pairs, from 200 to 500 base pairs.

### Optional Steps

According to a particular embodiment, the method of the invention further comprises at least a step of enzyme inactivation, performed after step (b) and/or after step (c). Particularly, step of enzyme inactivation may be performed between step (c1) and step (c2), and/or after step (c2). According to another particular embodiment, a step of enzyme inactivation is performed between step (b) and step (c1) and between step (c1) and step (c2).

Enzyme inactivation is used to stop any enzymatic activity. This is particularly suited when two successive steps involve different enzymes, to avoid that the prior enzyme activity interferes with the subsequent enzyme activity. Usual conditions for enzyme inactivation are well known by a skilled person. For example, an incubation at 65° C. for 20 minutes may be efficient to inactivate enzymes that have an optimal temperature of 37° C.

Additionally, or alternatively to the inactivation step, the method of the present invention may further comprise at least a step of nucleic acid purification, performed after step (b) and/or after step (c). Particularly, step of nucleic acid purification may be performed between step (c1) and step (c2), and/or after step (c2). According to another particular embodiment, a step of nucleic acid purification is performed between step (b) and step (c1) and between step (c1) and step (c2).

Nucleic acid purification is herein used to eliminate any enzymes present in the reaction medium between two successive steps. The purified nucleic acids are thus deprived of previous enzymes.

Nucleic acid purification can be performed by any conventional methods or by using any commercially available nucleic acid purification kit, for example a kit using filter plate, beads or paramagnetic beads which can bind with or retain DNA or RNA to separate nucleic acid from impurity.

In some embodiments, the method of the present invention further comprises a step (d) of error correction which is advantageously performed after step (c) or step (c2).

Error correction step aims to correct sequence error(s) that may occur during previous steps. The term “sequence error” refers to sequence mismatch(s), nucleotide insertion(s) or deletion(s) in one nucleic acid strand compared to predetermined sequence, etc.

In a particular embodiment, the step of error correction involves endonuclease-mediated error correction or a mismatch repair protein-mediated error correction. Examples of endonuclease that can be used for endonuclease-mediated error correction include, without limitations, T7 endonuclease, T4 Endonuclease VII, S1 nuclease, P1 nuclease or Cel I endonuclease. Examples of mismatch repair protein that can be used for mismatch repair protein-mediated error correction include, without limitation, MutS protein and its homologs, MutL protein and its homologs, or MutH protein. The mechanism of action of these enzymes and the sequence error correction methods using these enzymes are well known by the skilled person. For example, T7 endonuclease I-mediated and MutS-mediated error corrections are respectively described in Sequeira et al. (Mol Biotechnol (2016) 58:573-584) and Carr et al. (Nucleic Acids Res. 2004; 32(20): e162.).

According to an embodiment, a single or several successive error-correction steps can be performed.

In some embodiments, the method of the invention further comprises a step (e) of recovering the double-stranded nucleic acid. The recovery step may be performed by any standard nucleic acid purification method, for example by gel purification, column of affinity, or any commercially available nucleic acid purification kit, for example the kit using filter plate, beads or paramagnetic beads which can bind with or retain DNA or RNA to separate nucleic acid from impurity.

### Production of Double-Stranded Polynucleotide

It is a further purpose of the present invention to provide a method of producing a double-stranded polynucleotide with predetermined sequence. Said double-stranded polynucleotide of interest may be a fragment of genomic DNA, a gene of interest, a cDNA, etc. To this end, according to the present invention, two or more double-stranded nucleic acids, with different sequences, generated by the aforementioned method are further linked together to produce a double-stranded polynucleotide of interest.

In a particular embodiment, said double-stranded nucleic acids are linked to each other by a ligase or a polymerase.

The present invention thus provides a method for producing a double-stranded polynucleotide of interest, comprising the following steps:
generating at least two double-stranded nucleic acid by implementing at least steps (a) to (c) of above described method, said at least two double-stranded nucleic acids corresponding to at least two different fragments of polynucleotide of interest,
linking said fragments according to a predetermined order by use of a ligase and/or polymerase and/or chemical ligation to produce said polynucleotide.

In a particular embodiment, each double-stranded nucleic acid comprises at least at one end an overlap of sequence with another double-stranded nucleic acid.

In a particular embodiment, each double stranded comprises at least 4 to 40 nucleotides complementary overhang.

Said overlap of sequence guarantee each double-stranded nucleic acid to be joined in a correct order to produce the desired polynucleotide of interest. Then polymerase chain extension or other methods may be used for joining each double-stranded nucleic acid.

In a particular embodiment, double stranded DNA are devoid of homology and are connected one to the other with a small oligonucleotide of 30 to 70 nt that share homology to each adjacent double stranded DNA. The adjacent DNA can then be ligated together based on a ligase chain reaction.

### Applications of the Method

The use of the method of the present invention may significantly increase the performances of any process employing manipulations of nucleic acids. As nonlimiting examples, the use of the present invention is particularly advantageous in the following fields: preparation of genetic constructs, production of interfering RNA molecules, DNA or RNA chip production, construction of cell strains or lines, enzymatic engineering, development of protein models, development of biotherapies, development of animal or plant models.

The nucleic acids obtained with the method of the present invention exhibit a high degree of purity, allowing their direct use, i.e., without additional treatment stages.

If need be, the double-stranded nucleic acids or double-stranded polynucleotides of interest generated with the methods of the present invention can undergo additional targeted modifications. For instance, it is possible to circularize the nucleic acid fragments, to react the nucleic acid fragments with other chemical entities, etc.

### Kits

The present invention includes kits for carrying out methods of the invention. The term “Kit” refers to any delivery system for delivering materials or reagents for carrying out a method of the invention. In the context of reaction assays, such delivery systems include systems and/or compounds (such as dilutants, surfactants, carriers, or the like) that allow for the storage, transport, or delivery of reaction reagents (e.g., nucleic acid, enzymes, fluorescent labels, such as mutually quenching fluorescent labels, fluorescent label linking agents, quenching agents, etc. in the appropriate containers) and/or supporting materials (e.g., reaction medium, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for one or some steps of the method, while a second or more containers contain other one or more enzymes for other steps of the method.

In one aspect, said kit concerns a kit for generating a double-stranded nucleic acid having a predetermined sequence. In a particular embodiment, said kit concerns a kit for generating a double-stranded nucleic acid by enzymatic means.

According to a particular embodiment, said kit comprises:
A plurality of single-stranded oligonucleotides, a first hairpin-forming oligonucleotide comprising a single-stranded overhang and a second hairpin-forming oligonucleotide comprising a single-stranded overhang;
Optionally a first enzymatic reagent and/or a first reaction medium to be used for assembling the single-stranded oligonucleotides and a first and second hairpin-forming oligonucleotides into a closed double-stranded nucleic acid,
Optionally a second enzymatic reagent and/or a second reaction medium to be used for eliminating single-stranded oligonucleotide loops, intermediate products, unassembled single-stranded oligonucleotides and/or unassembled hairpin-forming oligonucleotides.

The first enzymatic reagent contains one or more ligases. Different ligases may be stored in different containers.

The second enzymatic reagent contains for example an exonuclease and a single-strand specific nuclease.

The kit of the present invention can further comprise a third enzymatic reagent and a reaction medium for sequence error correction. Said third enzymatic reagent contains for example an endonuclease and/or a MutS protein.

The kit of the present invention can also further comprise a system for nucleic acid purification, for example a column for purification by affinity, and a reaction medium.

In another aspect, the kit of the invention concerns a kit for producing a double-stranded polynucleotide of interest. Said kit comprise a kit as described before for generating a double-stranded nucleic acid having a predetermined sequence and a further enzymatic reagent and a reaction medium for assembling double-stranded nucleic acid. Said further enzymatic reagent contains for example a ligase which can ligate blunt-end double-stranded nucleic acid or a polymerase.

The present invention is illustrated more in detail by following examples.

### EXAMPLES

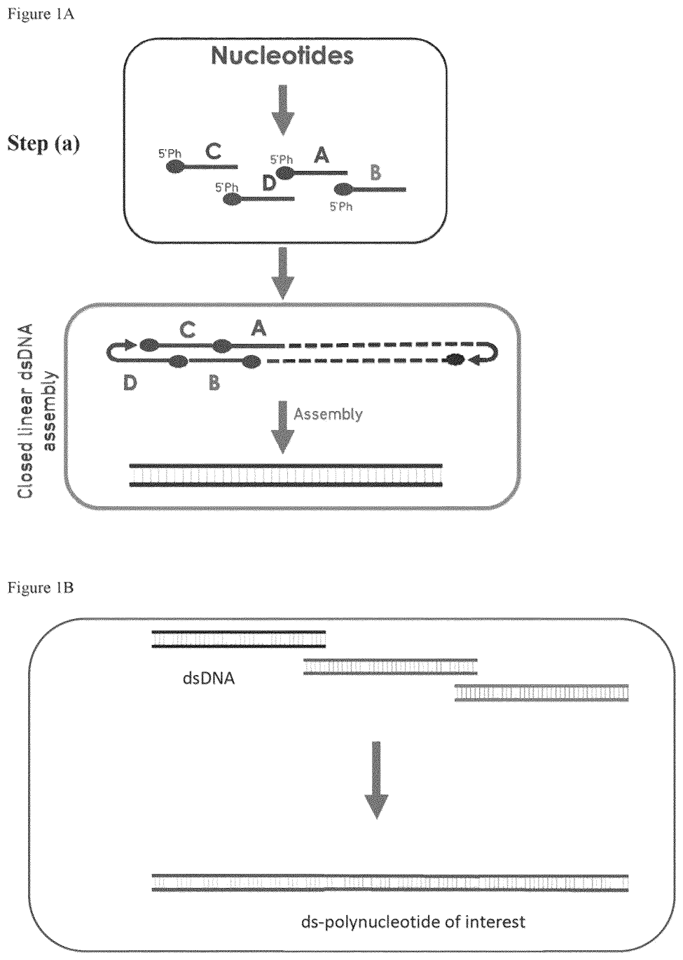

FIG. 1A illustrates a particular embodiment of the method of the invention for generating linear double-stranded nucleic acid, comprising:
enzymatic synthesis of 5′phosphorylated single-stranded oligonucleotides A, B, C, and 5′phosphorylated hairpin-forming oligonucleotides D,
   a segment of sequence of oligonucleotide B being complementary with a segment of sequence of oligonucleotide A, another segment of sequence of oligonucleotide B being complementary with a segment of sequence of oligonucleotide C, while another segment of sequence of oligonucleotide C being complementary with the sequence of overhang of hairpin-forming oligonucleotide D;
assembling of said single-stranded oligonucleotides and hairpin-forming oligonucleotides, to generate a closed double-stranded nucleic acid (step (a));
digesting single-stranded oligonucleotide loops to generate the linear double-stranded nucleic acid.

FIG. 1B illustrates a particular embodiment of the method of the invention for producing a double-stranded polynucleotide of interest: several double-stranded nucleic acids generated by the method illustrated in FIG. 1A are provided. Each double-stranded nucleic acid contains at least at one end an overlap of sequence with another double-stranded nucleic acid. These overlapping sequences are joined together by polymerase chain extension or other methods to produce a double-stranded polynucleotide.

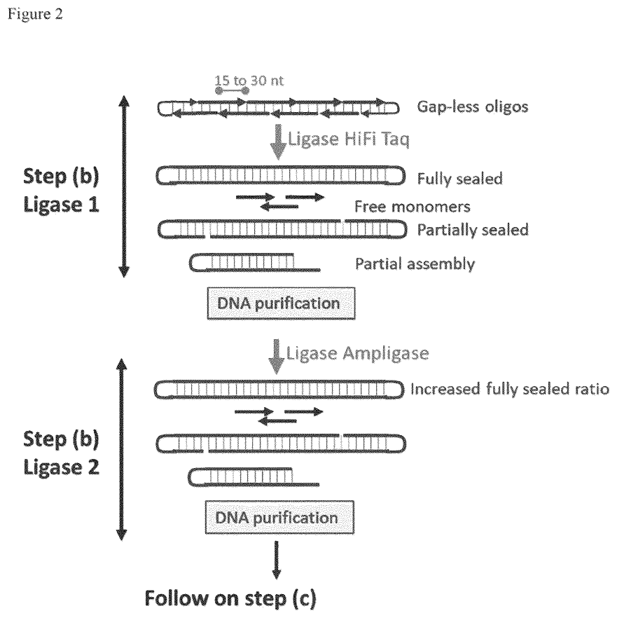

FIG. 2 illustrates step (b) of a particular embodiment of the method of the invention, wherein step (b) comprises:
a step of assembling single-stranded oligonucleotides and 2 hairpin-forming oligonucleotides by HiFi Taq ligase;
a step of DNA purification;
a step of further assembling unassembled monomers in the first ligation step and intermediate products obtained in the first ligation step by Ampligase to produce closed double-stranded nucleic acid;
a step of DNA purification.

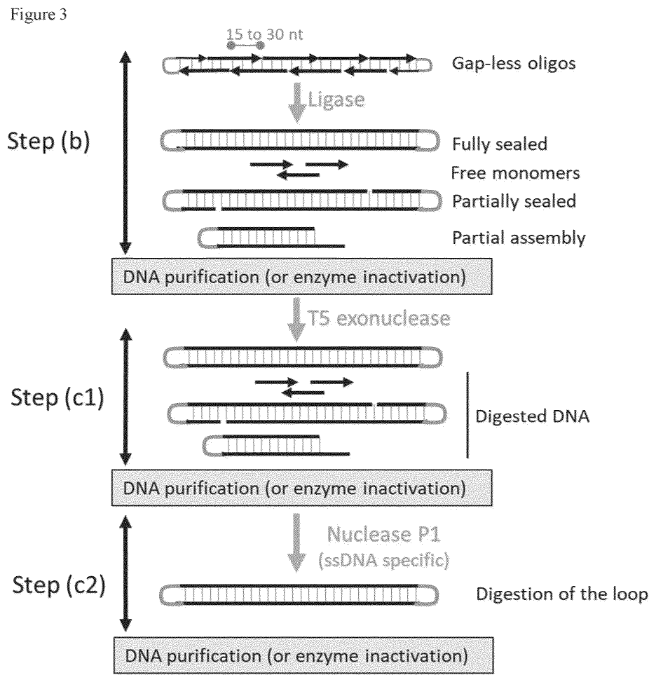

FIG. 3 illustrates another particular embodiment of the method of the invention which comprises successively:
step (b) of assembling several single-stranded oligonucleotides and 2 hairpin-forming oligonucleotides to produce a closed double-stranded nucleic acid;
a step of enzyme inactivation or DNA purification;
a step (c1) of digesting unassembled monomers and intermediate products with T5 exonuclease,
a step of enzyme inactivation or DNA purification;
a step (c2) of digesting the single-stranded loops of the closed double-stranded nucleic acid with nuclease P1;
a step of enzyme inactivation or DNA purification.

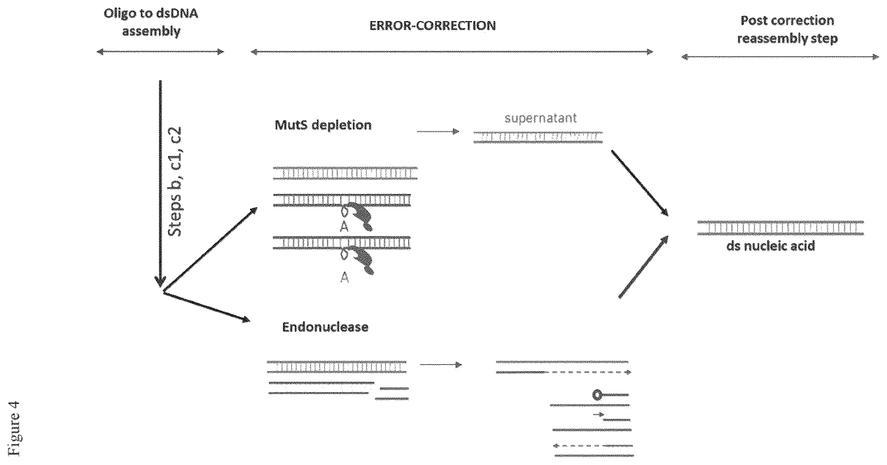

FIG. 4 illustrates another particular embodiment of the invention, wherein a step of error-correction is performed after the generation of linear double-stranded DNA. Said step of error-correction is carried out either by MutS depletion or by endonuclease-base cleavage. In MutS depletion, MutS protein recognizes and binds to the kink structure of a non-matching base on one strand. The MutS proteins bound to the mismatch-containing DNA can subsequently be trapped onto specific affinity-resin allowing thus the depletion of mismatch-containing dsDNA. In endonuclease-mediated error correction, mismatch specific endonucleases such as T7_endonuclease recognizes the mismatched nucleotide and cleaves the double-stranded DNA near the error. The perfect match dsDNA remain unaffected by the T7-endonuclease and can be used by a polymerase as a template to repair the mismatch-cleaved product. MutS and endonuclease-mediated error-correction can be used individually or in combination to significantly decrease the error-rate of the final dsDNA.

### Example 1: Oligos Ranging From 30 to 60 nt Long Allow the Assembly Of GFP Sub-Domains

The following is an example that uses the loop assembly method of the present disclosure to generate a synthetic construct composed of the green fluorescent protein (GFP) placed under the control of an in vitro transcription-translation coupled system. The complete sequence is an 820 base pairs dsDNA fragment.

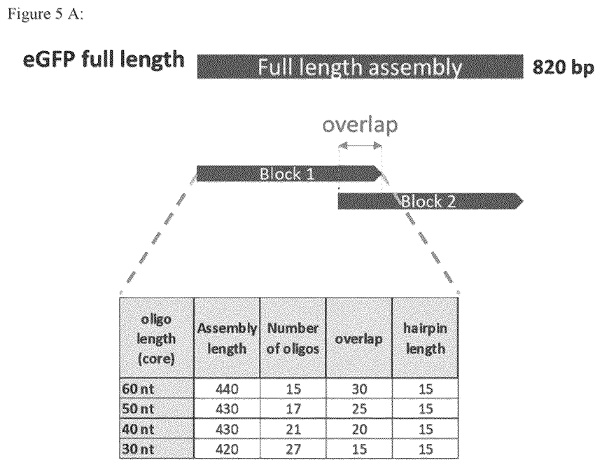

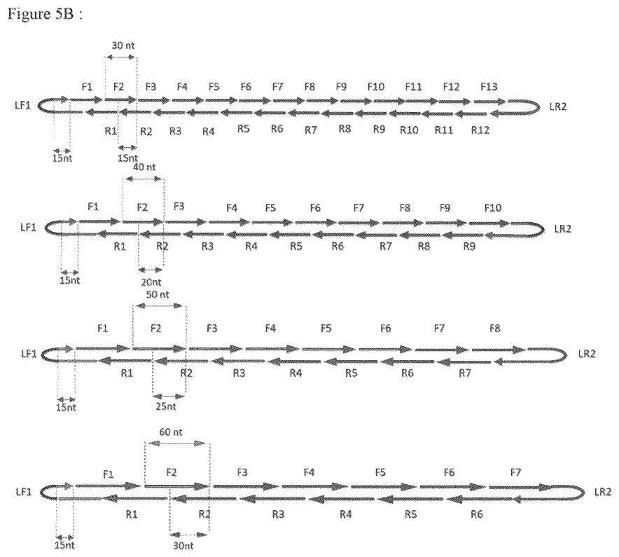

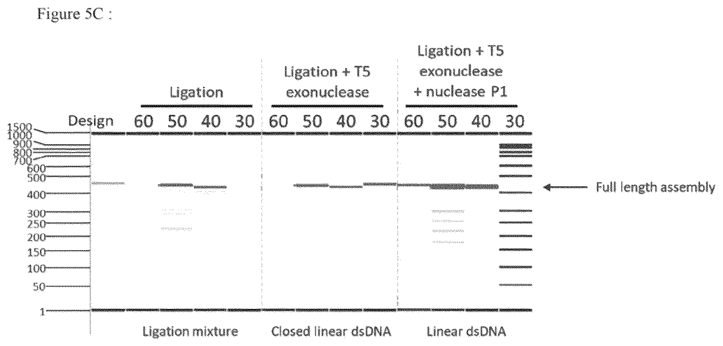

First the complete sequence was split into 2 overlapping domain (FIG. 5A). The loop assembly method described here has been used to generate the 5′ domain of the synthetic GFP construct.

The assembly target molecules are composed as follow (FIG. 5B):
30 nt design: 420 base pairs final assembly composed of 2 hairpins forming oligos of 55 nt in length and 25 core oligonucleotides of 30 nt in length.
40 nt design: 430 base pairs final assembly composed of: 2 hairpins forming oligos of 55 nt in length and 19 core oligonucleotides of 40 nt in length
50 nt design, 430 base pairs final assembly composed of: 2 hairpins forming oligos of 60 nt in length and 15 core oligonucleotides of 50 nt in length.
60 nt design, 440 base pairs final assembly composed of: 2 hairpins forming oligos of 60 nt in length and 11 core oligonucleotides of 60 nt in length and 2 core oligonucleotides of 55 nt in length

First, the oligonucleotides required for the loop assembly have been designed according to the 4 different core oligonucleotides length: 60 nt, 50 nt, 40 nt and 30 nt. The hairpin forming oligonucleotides have been designed with a hairpin domain of 15 nt, a loop of 5 nucleotides and an overlap to the adjacent core oligo of 15 nt for the 30 nt design; of 20 nt for the 40 nt design; of 25 nt for the 50 nt and 60 nt designs. The core oligonucleotides have a length of 30 nt, 40 nt 50 nt for the respective design 30 nt, 40 nt, and 50 nt. For the 60 nt design, the forward oligonucleotide that overlap to the forward loop and the reverse oligonucleotide that overlap with the reverse loop have a length of 55 nt while the other oligonucleotides are 60 nt in length (FIG. 5B).

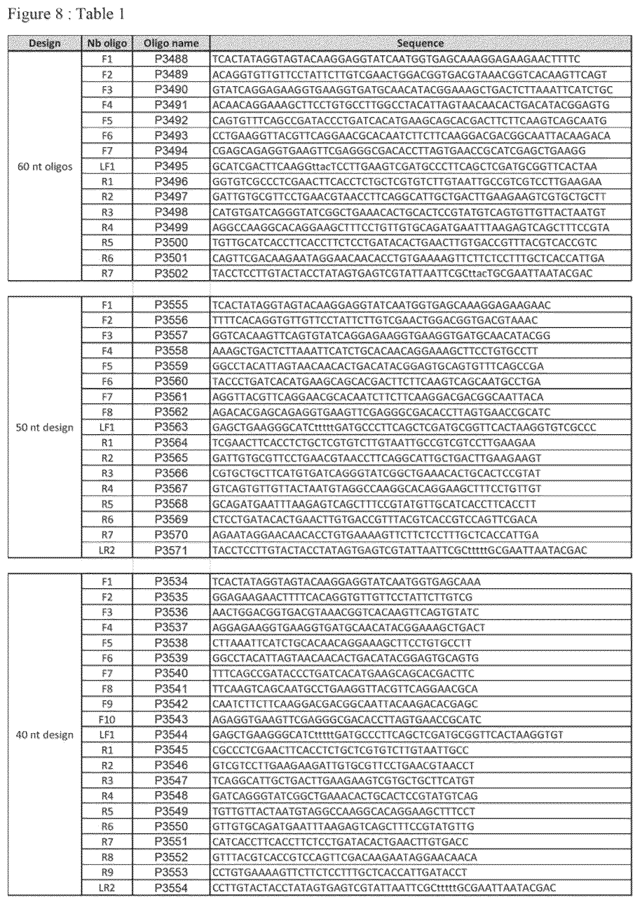

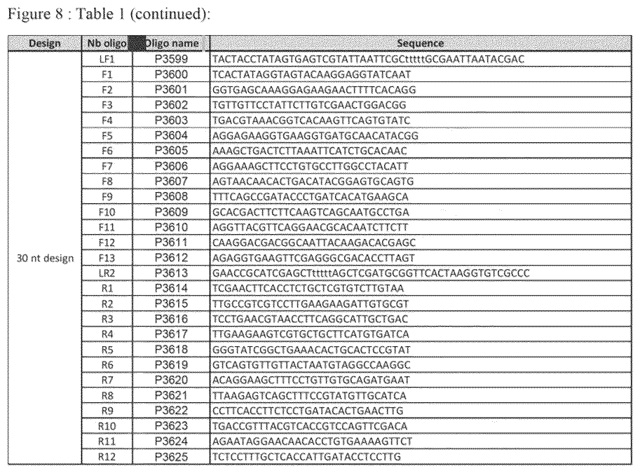

Each oligonucleotide has been synthesized with a 5′phosphate modification. List of oligonucleotides is found in table 1 (FIG. 8).

The oligonucleotides composing each of the assembly have been pooled together in an equimolar ratio. Each assembly reaction is performed with 0.048 mM (2.86 pmol) or 0.097 mM (5.84 pmol) final concentration of each oligonucleotide. Ligation reaction is performed in a final volume of 60 mL in presence of 5% PEG 8000 final concentration and 1 mL HiFi Taq DNA ligase (NEB). HiFi Taq DNA ligase is a thermostable ligase that allows to proceed with repeated cycle of ligation as follow: initial denaturation at 94° C. for 1 minute, 50 cycles of ligation composed of (10 seconds denaturation at 94° C., 30 seconds annealing at 55° C., 1 minute ligation at 60° C.).

Upon ligation completion, samples are purified on DNA purification column following manufacturer's instruction (Monarch PCR & DNA cleanup Kit, NEB) except that the elution is performed in 35 mL of molecular biology grade water.

Analysis of the elution on capillary electrophoresis reveals the presence of the full length closed linear dsDNA assembly as well as many partial ligation products ranging from 50 to 400 base pairs (FIG. 5C, Ligation samples).

The non-closed forms of DNA are eliminated by treatment with the T5-exonuclease that initiates degradation at the 5′ termini of linear or nicked double-stranded DNA in the 5′ to 3′ direction. The purified ligated samples are incubated for 30 min at 37° C. in presence of 10 units of T5 exonuclease (NEB) in a buffer containing 50 mM potassium acetate, 20 mM tris-acetate, 10 mM magnesium acetate, 1 m M DTT at pH 7.9 (buffer 4, NEB). Upon incubation completion, the digestion is purified on DNA purification column following manufacturer's instruction (Monarch PCR & DNA cleanup Kit) except that the elution is performed in 35 mL of molecular biology grade water.

Analysis of the elution on capillary electrophoresis reveals the presence of a unique form of DNA corresponding to the expected full-length assembly (FIG. 5B, ligation+T5 exonuclease samples). This DNA is a closed circular DNA form.

Cleavage and reversion of the hairpin is performed by digestion of the loop with the nuclease P1 (NEB) which is a single-strand specific endonuclease. The cleanup elution of the T5 exonuclease reaction is incubated for 30 min at 37° C. in presence of 10 units of nuclease P1 in the following buffer: 1 0 mM Bis-Tris-propane pH 7.0, 10 mM MgCl2, 100 ug/ml BSA (NEB buffer 1.1). Upon completion, the digestion has been purified on DNA purification column following manufacturer's instruction (Monarch PCR & DNA cleanup Kit) except that the elution is performed in 35 mL of molecular biology grade water.

Analysis of the elution on capillary electrophoresis reveals the presence of a unique form of DNA corresponding to the expected full-length assembly (FIG. 5B, ligation+T5 exonuclease+nuclease P1 samples). This DNA is a linear dsDNA composed of the target sequence of assembly.

The disclosed method allows the recovery of purified full-length dsDNA of expected sequence starting from single-stranded oligonucleotides of 30 to 60 nt long.

Elution can optionally be treated with the mung bean nuclease to remove traces of loop scar. The mung bean nuclease is single stranded specific endonuclease that catalyzes the removal of single-stranded extension in double-stranded DNA. The elution is incubated in presence of 0.1-1 unit of mung bean nuclease for 15 minutes at 30° C. in the following buffer: 30 mM NaCl, 50 mM sodium acetate, 1 mM ZnSO4, pH 5.

### Example 2: Loop Removal With Nuclease P1

This example demonstrates that the hairpin-containing oligonucleotides prevent degradation of the closed linear double stranded-DNA from exonuclease digestion and can be subsequently reverted with nuclease P1 digestion to release pure full-length linear dsDNA devoid of partial assemblies.

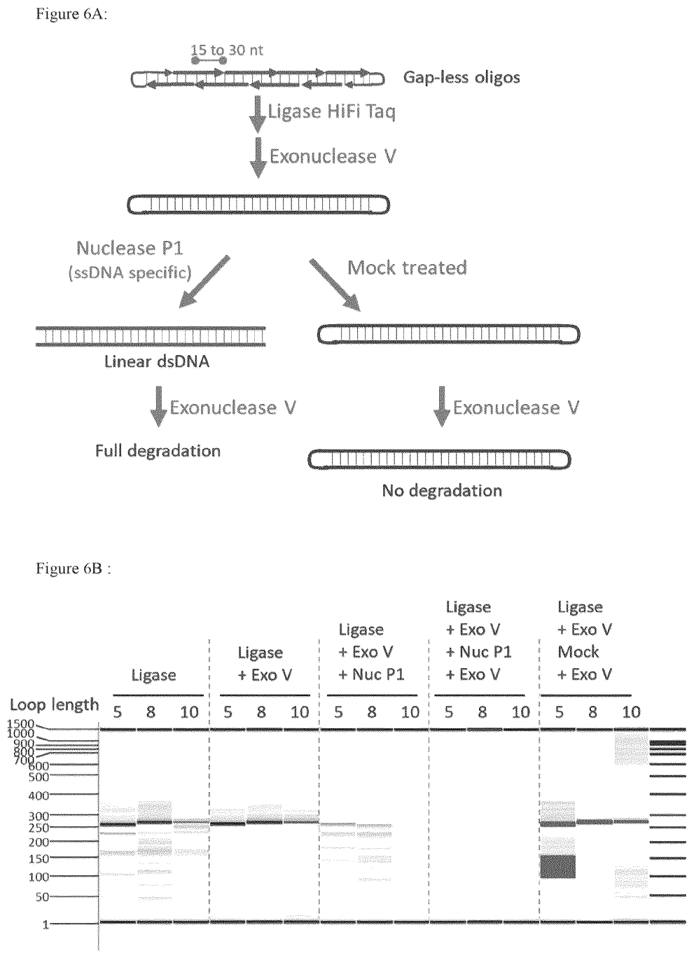

Assemblies have been designed with 60 nt long core oligonucleotides and hairpin-containing oligonucleotides containing a loop domain of 5, 8 or 10 nucleotides long. Oligos have been ligated together as described in example 1, and subsequently digested with exonuclease V. The exonuclease V is an exonuclease that degrades the ssDNA and dsDNA starting from 5′ or 3′ ends. In contrast to T5 exonuclease, the exonuclease V cannot initiate the degradation from a nicked DNA. When treated with exonuclease V, all partial assemblies are degraded, and one detects the full-length forms of dsDNA with some additional slower migrating forms that reflect nicked DNA. Upon subsequent digestion with the nuclease P1, because of the loop digestion, the molecules are running with the same apparent length (FIG. 6A and 6B).

When these samples are subsequently submitted to the exonuclease V, molecules are fully digested to monomers (FIG. 6B, lanes labelled ligase+Exo V+NucP1+Exo V). This demonstrates that the loops of hairpin-containing oligonucleotides have been digested so that the DNA becomes accessible to the exonuclease V. This is directly linked to the nuclease P1 treatment since a sample for which the nuclease P1 has been substituted with water (mock treated sample, FIG. 6A) is resistant to the subsequent exonuclease V treatment (FIG. 6B, lanes labelled ligase+Exo V+Mock+Exo V).

### Example 3: DNA Assembled With the Present Disclosure Are Compatible With Standard Molecular Biology Assay

Assembly of oligonucleotides into dsDNA allows the creation of de novo DNA sequences, without preexisting template. Such DNA are useful for subsequent reactions such as cloning, amplification or further reassembly into longer DNA.

To prove the compatibility of such synthetic DNA with standard biology process, two synthetic DNA constructed according to the method described in example 1 have been reassembled together based on polymerase overlap extension to create the full-length gene.

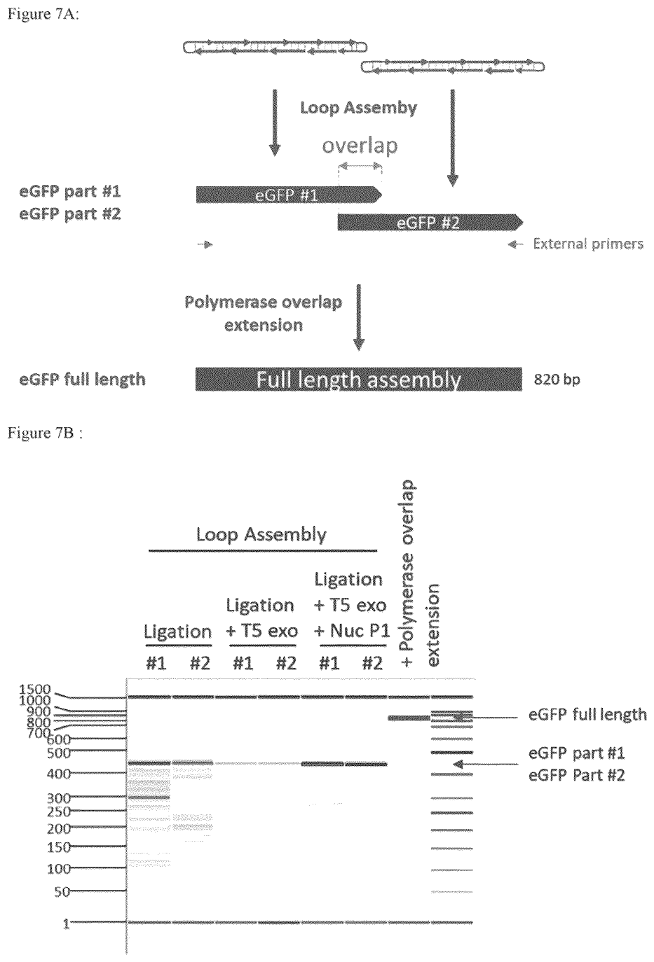

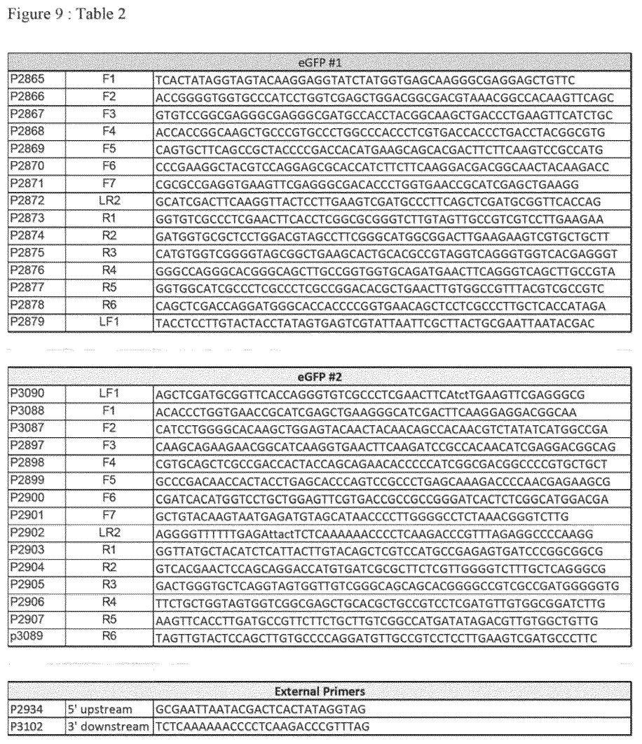

The eGFP synthetic construct has been chosen as assembly target, the full-length gene is 820 base pairs long. Full-length eGFP has been assembled into 2 dsDNA of 440 base pairs each, with an overlap of 60 nt long (FIG. 7A). eGFP #1 and eGFP #2 have been assembled starting from oligonucleotides of 60 nt long as described in example 1. List of oligonucleotides composing part #1 and part #2 is described in table 2 (FIG. 9). Following nuclease P1 digestion, the DNA have been cleaned up on column and eluted into 50 mL of molecular biology grade water.

The eGFP part #1 and part #2 have been submitted to overlap extension assay based on a commercial kit (Watchmaker Genomics Gene Assembly PCR Kit, WatchMaker Genomics Inc). 15 mL of eluted eGFP part 1 and 15 mL of eGFP part 2 have been mixed in the assembly reaction in presence of 300 mM final concentration of each external primer. Assembly reaction has been performed according to provider's recommendation using the provided polymerase, polymerase buffer and DNTPs. Assembly and amplification have been performed with the following cycle profile: 95° C. for 3 minutes, 25 cycles of (95° C. 20 seconds, 62° C. for 30 seconds, 72° C. for 1 minute) followed by 1 minute final extension at 72° C.

All samples have been analyzed on capillary electrophoresis (FIG. 7B). After completion of the closed linear dsDNA assembly process, one can detect the two eGFP parts of 440 base pairs long each. Following polymerase overlap extension, a single form of 820 base pairs long is detected. This matches the intended full-length eGFP linear dsDNA sequence. The absence of eGFP part #1 and eGFP part #2 monomers confirms the functionality of the synthetic DNA created with present disclosure in standard overlap extension reaction.

1. A method for generating a double-stranded nucleic acid having a predetermined sequence, comprising the following steps:
(a) providing (i) a plurality of single-stranded oligonucleotides, (ii) a first hairpin-forming oligonucleotide comprising a single-stranded overhang and (iii) a second hairpin-forming oligonucleotide comprising a single-stranded overhang,
the sequence of each single-stranded oligonucleotide being at least partially complementary with the sequence of at least one other single-stranded oligonucleotide of said plurality, and/or with the sequence of the single-stranded overhang of the first or second hairpin-forming oligonucleotide,
(b) reacting said plurality of single-stranded oligonucleotide, the first and second hairpin-forming oligonucleotides with a ligase to form a closed double-stranded nucleic acid with two single-stranded oligonucleotide loops in a reaction medium, and
(c) subjecting the reaction product of step (b) to enzymatic, physical or chemical means to eliminate said two single-stranded oligonucleotide loops.

2. The method according to claim 1, wherein step (b) is performed with at least two different ligases.

3. The method according to claim 1, wherein said ligase is T4 ligase, Ampligase, HiFi Taq ligase, Pfu DNA ligase, 9° north ligase or any thermostable ligase that recognizes 2 adjacent nucleotides bridged by a complementary DNA/RNA strand.

4. The method according to claim 1, wherein step (c) comprises:
subjecting the reaction product of step (b) to a nuclease to digest the single-stranded oligonucleotide loops of the closed double-stranded nucleic acid.

5. The method according to claim 1, wherein step (c) comprises subjecting the reaction product of step (b) simultaneously to an a nuclease to eliminate two single-stranded oligonucleotide loops, and an exonuclease to eliminate the intermediate products, unassembled single-stranded oligonucleotide or unassembled hairpin-forming oligonucleotide.

6. The method according to claim 5, wherein said exonuclease is exonuclease V, exonuclease T5, nuclease BAL-31 or a cocktail of several enzymes selected from the group consisting of exonuclease III and Lambda exonuclease or exonuclease VII and exonuclease III.

7. The method according to claim 4, wherein said nuclease is a single-stranded specific endonuclease.

8. The method according to claim 1, wherein said plurality of single-stranded oligonucleotides comprises from 3 to 100 single-stranded oligonucleotides.

9. The method according to claim 1, wherein the sequence of each single-stranded oligonucleotide of said plurality comprises two segments, each segment being complementary with a segment of the sequence of another single-stranded oligonucleotide of said plurality, or with the full sequence of the single-stranded overhang of the first or second hairpin-forming oligonucleotide.

10. The method according to claim 1, wherein each single-stranded oligonucleotide comprises from 10 to 100 nucleotides.

11. The method according to claim 1, wherein the single-stranded overhang of the first or second hairpin-forming oligonucleotides comprises from 5 to 35 nucleotides.

12. The method according to claim 1, wherein said double-stranded nucleic acid has a length from 50 to 1000 base pairs.

13. A method for producing a double-stranded polynucleotide of interest, comprising the following steps:
generating at least two double-stranded nucleic acids by the method according to claim 1, said double-stranded nucleic acids corresponding to at least two different fragments of said polynucleotide, and
linking said fragments according to a predetermined order by use of a ligase and/or polymerase to produce said polynucleotide.

14. A kit for the implementation of the methods according to claim 1, comprising:
a plurality of single-stranded oligonucleotides, a first hairpin-forming oligonucleotide comprising a single-stranded overhang and a second hairpin-forming oligonucleotide comprising a single-stranded overhang.

15. The method of claim 1, wherein step (c) further comprises eliminating intermediate products, unassembled single-stranded oligonucleotide, or unassembled hairpin-forming oligonucleotides.

16. The method of claim 15, wherein step (c) comprises subjecting the reaction product of step (b) to an exonuclease to degrade the intermediate products, unassembled single-stranded oligonucleotides, or unassembled hairpin-forming oligonucleotides.

17. The method of claim 1, further comprising performing error correction step(s).

18. The method of claim 1, further comprising recovering the double-stranded nucleic acid.

19. The method according to claim 7, wherein said nuclease is selected from the group consisting of nuclease P1, Mung Bean nuclease and nuclease S1.

20. The method according to claim 1, wherein the double-stranded stem of the first or second hairpin-forming oligonucleotides comprises from 3 to 20 base pairs.
